(19) United States
(12) Patent Application Publication
(10) Pub. No.: US 2016/0324429 A1
(43) Pub. Date: Nov. 10, 2016

(54) Blood Pressure Cuff Pathogen Barrier

(71) Applicant: Pamela Maranville, Pacoima, CA (US)

(72) Inventor: Pamela Maranville, Pacoima, CA (US)

(21) Appl. No.: 15/046,273

(22) Filed: Feb. 17, 2016

Related U.S. Application Data
(60) Provisional application No. 62/117,359, filed on Feb. 17, 2015.

(51) Int. Cl. A61B 5/022

(52) U.S. Cl. CPC A61B 5/02233

(57) ABSTRACT

A sleeve forming pathogen barrier is provided which is configured for positioning between the surfaces of a blood pressure cuff and the skin of a patient. The sleeve is formed of a circular sidewall which may be rendered separable along an axial length whereby the sleeve will tear and allow removal of the sleeve from the arm of the patient, subsequent to completion of a blood pressure measurement by the cuff.

This application claims priority to U.S. Provisional Patent Application Ser. No. 62/117,359 filed on Feb. 17, 2015 which is included herein in its entirety by this reference thereto.

### BACKGROUND OF THE INVENTION

1. Field of the Invention

The present invention relates generally to blood pressure cuffs employed for ascertaining the blood pressure of a patient. More particularly, it relates to a device and method for positioning a pathogenic barrier, between an engaged blood pressure cuff, and the skin or clothing of a patient on which the cuff is engaged.

2. Prior Art

Measurements of the blood pressure of a human patient are conventionally achieved using devices which tightly engage a limb of the patient. In conventional medicine, in measuring a patient's blood pressure, the medical professional engages an inflatable cuff around the limb of the patient such as the upper arm. Once so engaged, a pump is employed to inflate the cuff to a maximum pressure level which is then gradually deflated while engaged around the patient's arm. This inflation enlarges the body of the cuff which decreases the interior circumference of the cuff while already tightly engaged around the limb of the patient. This results in the cuff circumferentially compressing upon the flesh of the patient it surrounds.

In this engaged position, the cuff is allowed to deflate over a period of time determined by the medical professional, or in some cases software running on an electronic blood cuff. Concurrent with the deflation of the cuff from this maximum pressure, a medical professional employs a stethoscope which is positioned on the person's skin at or adjacent to the site of the cuff engagement. With the stethoscope the medical professional listens to discern the sounds of a pulse of the patient during the duration of the slow deflation of the cuff, while concurrently viewing a pressure gauge. In this fashion the systolic and diastolic blood pressure level readings for the patient are ascertained when the medical professional views a pressure meter.

In recent years automatic blood pressure measuring devices have evolved to widespread use as they save professional time and in some cases allow for automated multiple measurements over time. Such automatic devices work in a similar fashion to the manual device in that once the inflatable cuff is properly positioned on a limb, the device will automatically inflate the cuff to compress it in position on the patient. The automated device employing software adapted to the task, will then use oscillations measured from sensors in the cuff or combinations of such oscillations and sounds from a microphone in or adjacent the cuff, to listen for a pulse. A pressure gauge continuously updates blood pressure to the automated device during deflation of the cuff. Thereafter software adapted to the task of measuring the oscillations, and/or sound of blood flow through blood vessels constricted by the cuff, will calculate the blood pressure levels of the patent. Heart rate is also generally calculated.

In both modes of blood pressure measurement, the medical implement of choice, is a non-disposable inflatable cuff which is engaged to the limb of each patient. The cuff surface thus comes in contact with either each patient's skin directly or a garment positioned in-between the skin and the cuff surface. However, it is preferred that the cuff be directly contact the skin surface for more accurate readings of pressure. Consequently, patients will frequently be asked to roll up their sleeve or pant leg during the procedure, to allow for a direct skin contact with the inflatable cuff.

While yielding valuable and accurate measurements of pulse and blood pressure levels, this procedure is problematic from the standpoint of the communication of pathogens such as viruses and bacteria and the like between patients. The cuff being inflatable and engaged with an expensive piece of equipment, is not disposable. It is therefore reused many thousands of times over the lifetime of each cuff. Further, due to the material forming the inflatable cuff and attached measuring device being plastic and cloth and combinations thereof, cuffs generally cannot be sterilized using high heat which would damage the cuff. Further, due to the nature of use of such cuffs for measuring blood pressure on a constant repeating basis, with multiple arriving patients daily, it would be inconvenient at best to remove each cuff, sterilize it, and reinstall it for each patient.

As such few if any blood pressure cuffs are decontaminated or sterilized after use even though such would help remove pathogens such as germs, viruses, and bacteria. Thus, in the conventional busy medical practice, patients, one after another, each with such pathogens on their skin and clothing, will have the blood pressure cuff placed in contact with their person for a reading of their blood pressure. This occurs within minutes in a busy medical practice or facility with a long stream of patients arriving daily.

Consequently, the conventional manner of use of such inflatable blood pressure measuring cuffs, can easily result in cross contamination and delivery to subsequent patients, the pathogens present on all of the previous patients on which the blood pressure cuff was employed. Thus, each sequentially subsequent patient from that of the first use of the cuff, is exposed to any pathogens communicated from the skin or person of every patient on which the cuff was previously engaged.

In a busy medical office or hospital, such use and constant engagement of the cuff on subsequent patients can expose subsequent patients on which the cuff is attached, to the pathogens communicated from the skin of hundreds or thousands of previous patients on which the cuff was engaged, should it not be at some point sterilized or otherwise decontaminated.

As can be discerned, conventional medical practice using blood cuffs to measure patient blood pressure, can result in the potential of thousands of infections being transmitted to patients, from a procedure developed to help keep them healthy.

As such, there exists an unmet need, for a device and method to prevent transmission of pathogens present on a blood pressure cuff, to patients on which it is engaged. Such a device and method, should not require removal and sterilization of a cuff which would not be an option in a busy medical practice. Such a device and method should provide a barrier between the skin and the person of a patient on which a pressure cuff is engaged, and the pathogens potentially positioned upon the surface of an inflatable blood pressure cuff. Such a device and method should be easily employable by the medical staff during the blood pressure measurement procedure to encourage use. Such a device should be inexpensive to also encourage use, due to the millions of blood pressure measurements performed and thereby render it easily adopted by medical providers at minimal cost. Further, such a barrier device should be both easily and quickly engaged as a barrier between the cuff and patient, and ideally, configured to prevent the patient and the medical professional from communicating pathogens to the patient during a removal of the barrier, subsequent to the procedure.

The forgoing examples of related art and limitations related therewith are intended to be illustrative and not exclusive, and they do not imply any limitations on the disclosed pathogen barrier invention and method described and claimed herein. Various limitations of the related art are already or will become apparent to those skilled in the art upon a reading and understanding of the specification below and the accompanying drawings.

### SUMMARY OF THE INVENTION

The device and method herein disclosed and described provides a solution to the shortcomings of measuring a patient's blood pressure using an inflatable cuff which is tightly placed in circumferential contact around an appendage such as a portion of an arm, leg, or finger. As noted, conventional inflatable cuffs provide no barrier between the exterior surface of the inflatable cuff, and the skin and person of a patient. Such contact with the skin or person of a patient can easily transfer patient-borne pathogens such as bacteria and viruses and the like, onto the surface of the inflatable cuff.

Consequently, each subsequent patient on which such a blood cuff is employed, is exposed to the sum of combined pathogens from every previous patient on which the inflatable cuff has been employed. As can be discerned, in a busy medical practice, clinic or hospital, such can amount to hundreds or thousands of previous patients who are both exposed to previously deposited pathogens, and, who concurrently communicate their own body-borne pathogens to take up residence on the inflatable cuff.

The device and method herein, provides a method and component forming a barrier between the skin of the patient being tested, and the inflatable cuff being employed for such blood pressure measurements. The disclosed device is configured and formed of material adapted to minimize or eliminate any interference with the results achieved during such a blood pressure test, while concurrently preventing transmission of pathogens from the patient to the inflatable cuff. Concurrently the device shields the skin and person of the patient, from communication and possible transmission of pathogens occupying space on a contaminated surface of the inflatable cuff.

In a preferred mode, the device is provided as individually employable barrier devices, which are easily dispensed from a roll of such barrier devices. Dispensing the device from a roll of such devices has been found to lessen any communication of pathogens to the device prior to positioning on the patient, when compared with a box holding a number of the devices. This is because, much like a box of disposable gloves, frequently users will reach into the box with bare hands to pull a device to be worn therefrom. In the process they contaminate the inside of the container and in many cases, a number of the housed gloves. Using a roll, only the barrier device to be engaged between the patient and cuff is contacted and removed, and this contact is with the exterior surface of the barrier which is maintained out of contact with the patient when operatively positioned.

The barrier device is formed of a thin polymeric material which has no pores or passages therein, to form a barrier blocking passage of pathogens, and may be configured in a number of modes and may either cover the entire arm or appendage of the patient, or, only an area exceeding a contact surface of the inflatable cuff with the skin of the patient. In cases where a disease which is highly contagious is known to be present, a barrier component which is elongated may be employed to cover the entire arm or leg of the patient if preferable. In most cases, a barrier component which covers the patient's skin or person, in an area exceeding that of the surface contact with the inflatable cuff, will be sufficient.

Experimentation has found that in a preferred mode, the barrier device should slide into position easily to encourage use and minimize time to engage it. However, once the measurement for blood pressure is complete, and the inflatable cuff removed, it has been found that is a frangible or a separable barrier device which may be easily torn off, may be superior to one that is removed by sliding it back off the arm or leg.

When the device is rendered frangible and removable by tearing or separation of the wall of forming the barrier device at a seam, fold, or thin wall section, it renders the device easily removed by the medical professional by simply pulling it apart to remove it from the patient. However, it has been found that while a circular barrier device which is slid off the arm or leg subsequent to use will still provide an excellent pathogen barrier. On some occasions the patient may attempt a self-removal and will contact the surface of the barrier which was previously in contact with the inflatable blood cuff. Such contact is not desirable and therefor the more preferred mode of the barrier device will either have a separable connection, or frangible line, or some other means for rendering two adjacent portions of the flexible sidewall forming the barrier device, frangible or separable, whereby it can be easily torn from the arm or leg.

Means for a separable engagement of the adjacent portions of the sidewall of the formed barrier device can include one or a combination of thinner sections of material, indentations in the material, perforations in the material, a rip cord engaged with the material and adapted to separate opposing sections, or an engageable seam where one side is complimentary to the other and may be disengaged such as by pulling on one or both opposing portions. In this fashion, once the measurement with the inflatable cuff is finished and the cuff removed, a tug upon one end of the barrier device will separate the sidewall forming it on opposing sides of a separation line, and cause it to dismount.

The material forming the barrier device is preferably a polymeric material or plastic material, which has no holes or gaps and is adapted to the task of preventing communication of pathogens between the patient and the inflatable cuff. Further, it can be extruded or moldable in a thin but strong sidewall forming the barrier component. Additionally, the material forming the device, can be impregnated prior to forming of the wall surface as above, using a pathogen deterring material such as silver ions or aluminum ions or a pharmaceutical which deters bacteria and viral growth on the surface.

Once removed from the patient by sliding or tearing with a separable sidewall, the device can be deposited for recycling since the extreme heat and pressure for extrusion and molding will kill such pathogens.

With respect to the above description, before explaining at least one preferred embodiment of the herein disclosed pathogen barrier for a surrounding inflatable blood cuff, and method thereof in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other pathogen shielding structures, methods and systems for carrying out the several purposes of the present disclosed device and method. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It is an object of the invention to provide a removably positionable shield component forming a barrier between an inflatable blood pressure cuff encircling a patient's arm or leg, and the skin or person of the patient.

It is an object of the invention to provide such a shield component which is easily positioned prior to encirclement by the inflatable cuff, and easily torn or removed from the patient subsequent to removal of the inflatable cuff.

It is yet another object of this invention to provide such a barrier device for a blood cuff, which is easily dispensed in a manner to avoid pre contamination, and which may be recycled.

These and other objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features of the inflatable blood cuff barrier and method herein. It is intended that the embodiments and figures disclosed herein are to be considered illustrative rather than limiting. In the drawings:

FIG. 1 depicts an elongated mode of the sleeve or device, shown in an as-used position, encircling the exterior of an arm or appendage of a patient and sandwiched in between the surface of the arm and a blood pressure cuff engaged thereround, and forming a barrier to contact from the pressurized blood pressure and communication of pathogens therethrough.

FIG. 2 shows a mode of the device similar to that of FIG. 1, however, with the device formed in a length which exceeds a width of the wall of the inflatable cuff contacting the patient.

FIG. 3 shows a mode for storing and dispensing the barrier devices herein disclosed and described by forming them to individual barrier devices, each of which may be sequentially torn from a roll thereof, without touching the others.

FIG. 4 is a view of the short mode of the sleeve or device such as in FIG. 2, without an axially positioned separable connection such as thinning or dimpling of the material or perforation, rip cord, or engageable edges employed in other modes.

FIG. 4a shows the separable line running between opposite edges of the sidewall forming sleeve or device herein formed using an adhesive strip to engage the sidewall, and shows the total barrier formed by the polymeric material which has no holes or passages where liquid or pathogens can communicate therethrough.

FIG. 4b shows a separable line running along the axial length of the formed sleeve or device, formed by a thinning of the polymeric material forming the device.

FIG. 4c shows the separable line running axially along the sidewall of the device using an overlap of opposing sides with adhesive therebetween.

FIG. 4d depicts a separable line running axially along the sidewall forming the sleeve or device herein, using a drawstring which is placed into the sidewall materially axially during extrusion.

FIG. 5 is a perspective view of another mode of the device of FIG. 1, with an axial separable line from a thinning or dimpling of the wall surface along the separable line formed in the sidewall defining the body of the device, and an end which may be sealed or open as with other longer modes.

As noted, in the especially preferred modes of the device 10 which are adapted for easy engagement and subsequent disengagement from the as-used position, a separable engagement between adjacent portions of the sidewall 12 defining a body of the barrier device 10 is provided. Such a separable engagement should still provide a barrier to pathogen communication during encirclement by the cuff 16. However, upon removal of the cuff 16, providing a separable engagement along the axial length of the device 10 allows for easy removal without the need to slide the arm or leg or appendage from the axial passage thereof. Such a separable engagement can include one or a combination of thinning of the wall of the material, perforations in the material, a rip cord engaged with the material to separate opposing sections when pulled, or an engageable seam where one side is complimentary to the other and may be disengaged such as by pulling on one or both opposing portions. In this fashion, once the pressure measurement with the inflatable cuff is finished, and the cuff removed, a tug upon one end of the barrier device will separate the sidewall forming it on opposing sides of a separation line, and cause it to easily dismount.

Other aspects of the present blood cuff shield and method thereof shall be more readily understood when considered in conjunction with the accompanying drawings, and the following detailed description, neither of which should be considered limiting.

### DETAILED DESCRIPTION OF THE PREFERRED EMBODIMENTS OF THE INVENTION

In this description, the directional prepositions of up, upwardly, down, downwardly, front, back, top, upper, bottom, lower, left, right and other such terms refer to the device as it is oriented and appears in the drawings and are used for convenience only and they are not intended to be limiting or to imply that the device has to be used or positioned in any particular orientation.

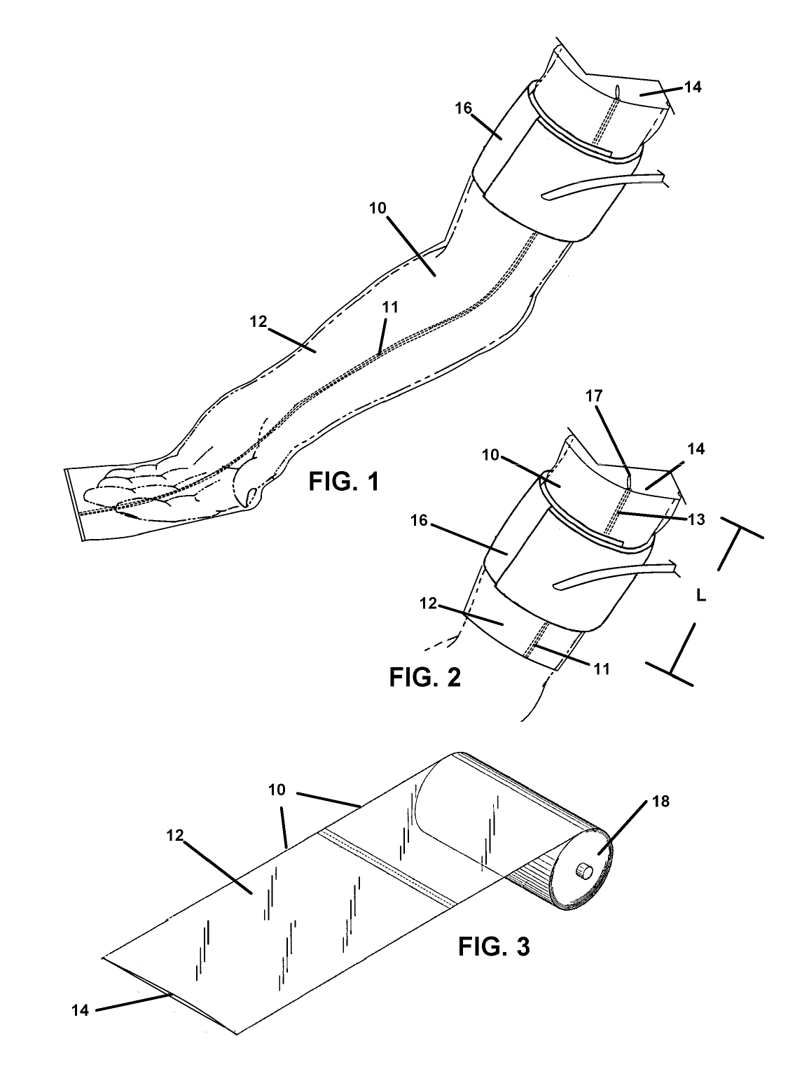

Now referring to drawings in FIGS. 1-5, wherein similar components are identified by like reference numerals, there is seen in FIG. 1 the noted elongated mode of the device 10. An as-used positioning is shown in FIGS. 1-2 and 4-5, wherein the circular sidewall 12 forming a sleeve, or herein the device 10, is positioned to encircle the arm or leg or appendage of a patent, at least in the area where a blood cuff 16 is engaged. This encirclement is accomplished by a sliding of the arm or leg through an axial cavity 14 defined by the interior surrounding surface of the sidewall 14, which is dimensioned to accommodate the arm or other body appendage therein.

So positioned, with the interior surface of the device 10 defining the axial cavity 14 in contact with the skin, clothing, or person of the patient, the sidewall 12 forms a physical barrier to any contact by the skin or person of the patient, with any surface of the compressed pressurized blood cuff 16, when operatively engaged. The polymeric material forming the sidewall 12 is solid and has no passages or gaps and therefore by barrier is meant that when positioned between the surface of a blood pressure cuff 16 and the skin of the patient, that no liquids, dirt, or pathogens such as bacteria, viruses, or germs, can pass through this formed barrier.

Also shown in FIG. 1 is a separable line 11, which in a particularly preferred mode of the device 10, runs the length of the device 10, from a first end adjacent the cuff 16 to an opposite second end, as shown in FIG. 1 and also in FIGS. 2 and 5. Such an inclusion will thereby allow for easy removal of the device 10 from the patient once testing is completed and the cuff 16 removed by a pulling of the sidewall 12 on one side of the separable line 11, causing the sidewall to rupture and open along the length of the separable line 11.

As noted, while the device 10 provides a significant benefit to prevent passing of pathogens between patients with or without the inclusion of the separable line 11, without the separable line 11, the device 10 will be slid off of the arm or leg. Such an action, especially in the full length versions of the device 10 of FIGS. 1 and 5, while an improvement is slightly less preferred as it may cause the exterior surface which might have been contaminated by contact or proximity to the cuff 16, to touch the patient during removal.

However, with the easy separation of the circular sidewall 12 along the separable line 11 shown, a pulling on the sidewall 12 on one side or the other of the separable line 11, will cause a tear or separation depending on how the sidewall joins at the separable line. This makes it easy for the medical professional to simply grasp the sidewall 12 adjacent the intersection of the separable line 11 with an end or edge, and pull to separate the opposing sides of the sidewall 12 to remove it from the patient.

As noted herein, the separable line 11 can be formed by a mechanical engagement of the opposing portions of sidewall 12 along the separable line 11, an adhesive engagement, an extremely small perforation, or a thinning of the sidewall itself in the area along the separation line rendering it easily torn. Other separable portions of the sidewall along a separation line allowing for the sidewall 12 to separate when pulled as would occur to those skilled in the art might be employed and are considered within the scope of this patent.

Additionally, as shown in FIG. 2, a drawstring 13 can be extruded into the sidewall 12, which when pulled upon such as with at a tab 17, causes a separation along the separable line 11, whereby the patient can remove their arm or leg through the formed gap. During extrusion the sidewall 12 is attached to opposing sides of the drawstring 13, and is easily torn along the separable line 11 by pulling the drawstring 13 in a direction normal to the plane of the sidewall 12.

As also can be discerned in FIG. 2, the device 10 may be formed in a shorter axial length than the depicted device 10 of FIG. 1 covering the entire appendage of the patient, and as shown in FIG. 5. In this abbreviated mode, the sleeve or device 10 is formed with an axial length “L” running from a first end to an opposite second end, which exceeds a width of the encircling inflatable cuff 16 and thereby forms the barrier to direct physical contact and pathogen transmission between the patient and the surface of the inflatable cuff 16. Currently a length “L” between 4 inches to 12 inches in the shorter mode of figure two, and an axial length of 12 to 25 inches for the longer mode as in FIG. 1, has shown to provide sufficient extension on each side of an engaged cuff 16 to afford protection from contact with the skin by the cuff 16.

In all modes of the device 10, the circumference of the interior of the axial cavity 14 has been found to work well if formed to yield an interior circumference from 6 inches to 22 inches. The device 10 can be formed in circumference and axial length sizes to accommodate the arms of children to adults, and if formed in that manner, the interior circumference would be varied in the noted range above as would the axial length “L”. Current preferred polymeric materials include one or a combination of polymeric materials from a group including polyethylene, polypropylene, PVC, Polyethylene, Polystyrene, and Polyester. The thickness of the sidewall 12 has been shown to work well at between 0.2 mils to about 0.9 mils, to form the sleeve or device 10 which will not interfere whatsoever with blood pressure measurements, and when formed to easily separate the sidewall 12 along a separable line 11, takes minimal force to separate when pulled.

This short mode of the device 10 has been shown to be more easily positionable to the as-used position to cover the skin and person of the patient at the site where the cuff 16 is encircled and inflated. Such would work well dispensed in a roll as in FIG. 3, or from boxes having multiple components of the device 10 therein. Alternatively, for the smaller mode such as in FIG. 2, the device 10 could be packaged in sterile packaging which can be opened to use the device on the patient.

Shown in FIG. 3 shows a mode for storing and dispensing the barrier devices 10. During manufacture, the individual devices 10 are formed in a roll 18 which dispenses individual devices 10 sequentially from one end of the roll 18. Perforations or other means for rendering individual sleeves or devices 10 separable from adjacent devices 10 on the roll, are employed. This mode of dispensing would work well adjacent blood pressure testing station whether in an automatic pressure testing device in a drug store, or a medically staffed blood pressure testing area of a medical facility. If employed in a public situation where such devices are automatic and provided for customer use, the exterior of the sidewall 12 forming the sleeve or device 10, could include coupons printed thereon employable for purchases in the store.

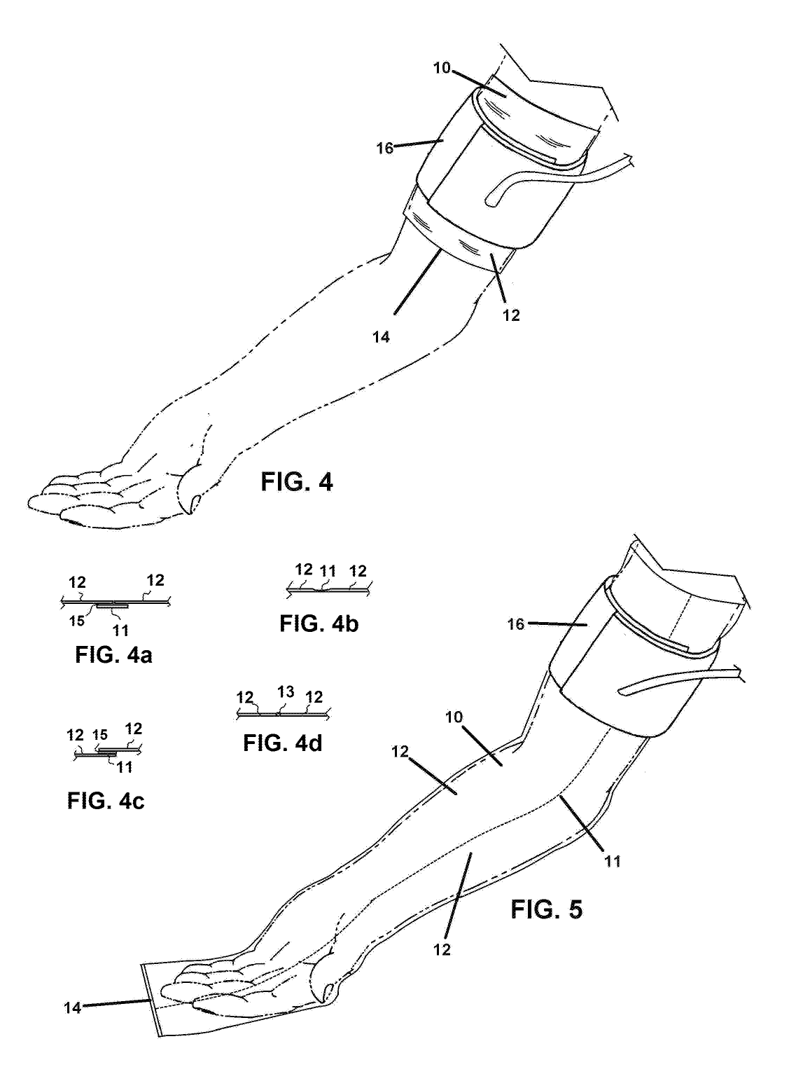

FIG. 4 is a view of the short mode of the device 10 such as in FIG. 2, but without an axially separable line 11 or portion. This mode simply slides off the arm of the patient once the cuff 16 is removed.

As noted herein, the separable line 11 running axially along the sidewall 12 forming the device 10, when present, allows for a separation of the sidewall along the separable line 11 for removal from the arm of the patient after use. Examples of such, which should in no manner be assumed as limiting, include the separable line 11 of FIG. 4a where two edges of the sidewall 12 are engaged with adhesive 15 using overlain removable adhesive strip; or a formation of the separable line 11 by a thinning of the polymeric material forming the sidewall 12 as in FIG. 4b; or an overlap of opposing edges of the sidewall 12 with adhesive 15 therebetween as in FIG. 4c; or a positioning of a drawstring 13 which is placed into the sidewall 12 axially during extrusion.

FIG. 5 is a perspective view of another mode of the device 10 such as in FIG. 1, with the formed axial separable portion running along the separable line 11 such as a thinning of the side wall 12 surface to form a separable or frangible line in the sidewall 12. The sidewall 12 as noted may be “thinned” along the line or in a very small perforated or dotted line pattern, to allow for an easy separation of opposing sides of the sidewall 12, on each side of the separable line 11. This will avoid holes in the sidewall 12 and maintain a barrier between the patient and the surface of the cuff 16 while allowing for an easy tearing of the sidewall along the line 11 for a removal of the device 10 from the patient, subsequent to completion of the test and removal of the cuff 16. In all modes of the device 10, the separable line 11 may be marked with ink or coloration or other indicia (not shown) such that the medical professional will know where to grasp the sidewall 12 to tear it along the separable line 11 to remove the device 10.

While all of the fundamental characteristics and features of the blood cuff shield system and method of employment have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed:

1. A pathogen barrier configured for positioning between a blood pressure cuff and the skin of a patient, comprising:
a sidewall of polymeric material forming a sleeve, said sleeve having an axial cavity;
said sleeve having a length running from a first end at one end of said axial cavity to a second end of said sleeve adjacent an opposite end of said axial cavity; and
said axial cavity dimensioned for insertion of an arm of a patient therein, during location of said sleeve to an as-used position with said sidewall encircling an appendage of said patient and sandwiched in-between said appendage and a blood pressure cuff engaged thereround, and
said sleeve in said as-used position, forming a barrier between said appendage and said blood pressure cuff encircling said appendage, said barrier preventing passage of any pathogens therethrough.

2. The pathogen barrier of claim 1, wherein said appendage is an arm of said patient.

3. The pathogen barrier of claim 1, wherein said axial cavity has a circumference from 6 inches to 22 inches.

4. The pathogen barrier of claim 2, wherein said axial cavity has a circumference from 6 inches to 22 inches.

5. The pathogen barrier of claim 1, wherein said length of said sleeve is between 4 to 12 inches.

6. The pathogen barrier of claim 2, wherein said length of said sleeve is between 4 to 12 inches.

7. The pathogen barrier of claim 3, wherein said length of said sleeve is between 4 to 12 inches.

8. The pathogen barrier of claim 4, wherein said length of said sleeve is between 4 to 12 inches.

9. The pathogen barrier of claim 1, wherein said length of said sleeve is between 12 to 25 inches.

10. The pathogen barrier of claim 2, wherein said length of said sleeve is between 12 to 25 inches.

11. The pathogen barrier of claim 3, wherein said length of said sleeve is between 12 to 25 inches.

12. The pathogen barrier of claim 4, wherein said length of said sleeve is between 12 to 25 inches.

13. The pathogen barrier of claim 1, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said appendage therethrough.

14. The pathogen barrier of claim 2, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.

15. The pathogen barrier of claim 4, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.

16. The pathogen barrier of claim 6, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.

17. The pathogen barrier of claim 8, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.

18. The pathogen barrier of claim 10, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.

19. The pathogen barrier of claim 12, additionally comprising a separable portion formed in said sidewall along a line running said length of said sleeve; and
whereby force from a pulling of said sidewall adjacent said separable portion will tear said sidewall along said line thereby forming a gap in said sidewall for exiting of said arm therethrough.
